(19) United States
(12) Patent Application Publication
(10) Pub. No.: US 2006/0129244 A1
(43) Pub. Date: Jun. 15, 2006

(54) Expandable intervertebral spacer method and apparatus

(75) Inventor: Michael D. Ensign, Salt Lake City, UT (US)

Correspondence Address: SEED INTELLECTUAL PROPERTY LAW GROUP PLLC, 701 FIFTH AVE, SUITE 6300, SEATTLE, WA, 98104-7092, US

(73) Assignee: Alphaspine, Inc., Sarasota, FL (US)

(21) Appl. No.: 11/260,880

(22) Filed: Oct. 25, 2005

Related U.S. Application Data
(60) Provisional application No. 60/622,097, filed on Oct. 25, 2004.
(60) Provisional application No. 60/687,500, filed on Jun. 3, 2005.
(60) Provisional application No. 60/687,185, filed on Jun. 3, 2005.
(60) Provisional application No. 60/687,498, filed on Jun. 3, 2005.
(60) Provisional application No. 60/687,499, filed on Jun. 3, 2005.

(51) Int. Cl. A61F 2/44

(52) U.S. Cl. 623/17.16

(57) ABSTRACT

An expandable interbody spacer (IBS) device designed to restore the disc height between vertebral bodies. The expandable interbody spacer device has an integral, moveable expansion member or spreader, provided between two plates. The plates are connected by one or more connecting members that retain the plates in a position proximate to one another while allowing the plates to move from a first unexpanded position to a second expanded position upon activation of the expansion member. According to aspects of the invention, the interbody spacer device can be implanted in an unexpanded or collapsed configuration, and then expanded to full height by engaging the expansion member. In other embodiments, the interbody spacer device may take various forms, for example, it may be cashew, rectangular or annular.

### BACKGROUND OF THE INVENTION

1. Field of the Invention

The present invention is directed to an intervertebral spacer device, and more particularly, to an expandable intervertebral spacer device that may be applied to various existing surgical approaches, for example, posterior lumbar interbody fusion (PLIF), transforaminal lumbar interbody fusion (TLIF), anterior lumbar interbody fusion (ALIF), minimally invasive lumbar interbody fusion (MILIF), lateral interbody fusion, and oblique interbody fusion.

2. Description of the Related Art

The cervical and lumbar portions of the spine are frequently fused to treat instability and degenerative diseases of the spine. There are many diverse approaches and a variety of indications available for lumbar interbody fusion. Despite the diverse approaches and indications, however, each approach generally targets restoration of disc height.

Difficulty in restoring disc height has traditionally stemmed from the surgical procedure and the interbody implants that are used. According to one procedure, surgical instruments are inserted to determine the proper implant size. The surgical instruments are then removed to allow room for the implant; however, when the instruments are removed, the disc space collapses. After the surgical instruments are removed, the implant is impacted into the disc space. This serial insertion and removal of instruments and subsequent impaction of the implant results in increased risk of adverse effects.

More recently, with the evolution of surgical instruments and the demonstration of increased clinical benefits, minimally invasive surgical approaches have gained acceptance. Minimally invasive techniques prescribe a reduction in the number of instruments in the wound thus furthering the need for expandable implants to provide restored disc height.

Many have attempted to create implants that obviate the need for height restoring instruments-and the need for impaction of implants. Various implants have been developed that provide the ability to adjust the size of the implant after insertion, for example, Published U.S. Patent Application Nos. 2005/0021041 (Michelson); 2005/0010295 (Michelson); 2004/0162618 (Mujwid et al.); 2004/0127994 (Kast et al.); 2004/0059421 (Glenn et al.); 2003/0195631 (Ferree); 2003/0130739 (Gerbec et al.); 2003/0065396 (Michelson); 2002/0128713 (Ferree); U.S. Pat. No. 6,852,129 (Gerbec et al.); U.S. Pat. No. 6,835,206 (Jackson); U.S. Pat. No. 6,821,298 (Jackson); U.S. Pat. No. 6,773,460 (Jackson); U.S. Pat. No. 6,648,917 (Gerbec et al.); U.S. Pat. No. 6,595,998 (Johnson et al.); U.S. Pat. No. 6,562,074 (Gerbec et al.); U.S. Pat. No. 6,558,424 (Thalgott); U.S. Pat. No. 6,524,341 (Lang et al.); U.S. Pat. No. 6,436,140 (Liu et al.); U.S. Pat. No. 6,419,705 (Erickson); U.S. Pat. No. 6,395,034 (Suddaby); U.S. Pat. No. 6,200,348 (Biedermann et al.); U.S. Pat. No. 6,190,414 (Young et al.); U.S. Pat. No. 6,176,882 (Biedermann et al.); U.S. Pat. No. 6,117,174 (Nolan); U.S. Pat. No. 6,102,950 (Vaccaro); U.S. Pat. No. 6,080,193 (Hochshuleret al.); U.S. Pat. No. 5,980,522 (Koros et al.); U.S. Pat. No. 5,800,547 (Schafer et al.); U.S. Pat. No. 5,702,453 (Rabbe et al.); U.S. Pat. No. 5,554,191 (Lahille et al.); U.S. Pat. No. 5,522,899 (Michelson); U.S. Pat. No. 5,514,180 (Heggeness et al.); U.S. Pat. No. 5,171,278 (Pisharodi); and U.S. Pat. No. 4,863,476 (Shepperd), herein incorporated in their entirety by reference.

The result has been the creation of a plethora of complex and expensive implants; many require special tools, involve screws that frequently result in cross threading, or include pop-up ratchet configurations that may fail when loaded.

### BRIEF SUMMARY OF THE INVENTION

An expandable interbody spacer (IBS) device designed to restore the disc height between vertebral bodies is provided in accordance with the present invention. The expandable interbody spacer device is adapted for implanting between adjacent vertebral bodies of a human spine as a load-bearing replacement for a spinal disc. The expandable interbody spacer device has an integral, moveable expansion member or spreader, provided between two plates. The plates are connected by one or more connecting members that retain the plates in a position proximate to one another while allowing the plates to move from a first unexpanded position to a second expanded position upon activation of the expansion member. According to aspects of the invention, the interbody spacer device can be implanted in an unexpanded or collapsed configuration, and then expanded to full height by engaging the expansion member. In one embodiment, the interbody spacer device is machined such that space is left in the center of the device to receive BMP and morsalized bone to aid in fusion after implantation of the device. In other embodiments, the interbody spacer device may take various forms, for example, it may be cashew, rectangular or annular.

### BRIEF DESCRIPTION OF THE SEVERAL VIEWS OF THE DRAWING(S)

In the drawings, identical reference numbers identify similar elements or acts. The sizes and relative positions of elements in the drawings are not necessarily drawn to scale. For example, the shapes of various elements and angles are not drawn to scale, and some of these elements are arbitrarily enlarged and positioned to improve drawing legibility.

Various embodiments will now be discussed with reference to the appended drawings. It is appreciated that these drawings depict only typical embodiments and are therefore not to be considered limiting of scope.

FIG. 1 illustrates a top view of an expansion member of the interbody spacer device in accordance with principles of the present invention.

FIG. 2 illustrates a side view of the expansion member of FIG. 1.

FIG. 3 illustrates a side view of a body of the interbody spacer device in accordance with principles of the present invention.

FIG. 4 illustrates a top view of the interbody spacer device of FIG. 3.

FIG. 5 illustrates a cross-sectional view of the interbody spacer device taken along line 5-5 of FIG. 4.

FIG. 6A illustrates a top view of an exemplary interbody spacer device in an unexpanded state in accordance with principles of the present invention.

FIG. 6B illustrates a side view of the interbody spacer of FIG. 6A in an unexpanded state in accordance with principles of the present invention.

FIG. 7A illustrates a top view of an exemplary interbody spacer device in an expanded state in accordance with principles of the present invention.

FIG. 7B illustrates a side view of the interbody spacer device of FIG. 6A in an expanded state in accordance with principles of the present invention.

FIG. 8 illustrates a top, front isometric view of a cashew shaped interbody spacer device in accordance with principles of the present invention.

FIG. 9 illustrates a bottom isometric view of the cashew shaped interbody spacer device of FIG. 8.

FIG. 10 illustrates a top plan view of the cashew shaped interbody spacer device of FIG. 8.

FIG. 11 illustrates a top view of an alternative embodiment of an interbody spacer device in an unexpanded state.

FIG. 12 illustrates an expansion member configured as a dowel for use in the interbody spacer device of FIG. 11.

FIG. 13 illustrates a side view of an alternative embodiment of an interbody spacer device for restoring the lordodic angle in accordance with principles of the present invention.

FIG. 14 illustrates a side view of an alternative embodiment of an interbody spacer device having a wedge shaped expansion member in accordance with principles of the present invention.

FIG. 15 illustrates an end view of an alternative embodiment of an interbody spacer device, wherein the expansion member is aligned in the center of the device and connection elements are aligned along outer edges in accordance with principles of the present invention.

FIG. 16 illustrates a side view of an alternative embodiment of an interbody spacer device having separate spring members as the connection element in accordance with principles of the present invention.

FIG. 17 illustrates a top, front isometric view of a disc shaped cervical or anterior interbody spacer device having a superior tab in accordance with principles of the present invention.

FIG. 18 illustrates a top, front isometric view of a disc shaped cervical or anterior interbody spacer device without the superior tab in accordance with principles of the present invention.

FIG. 19 illustrates a top view of the disc shaped interbody spacer device of FIG. 17.

FIG. 20 illustrates an end view of the disc shaped interbody spacer device of FIG. 17.

FIG. 21 illustrates a side view of the disc shaped interbody spacer device of FIG. 17.

FIG. 22 illustrates a rear isometric view of a rectangular shaped interbody spacer device in accordance with principles of the present invention.

FIG. 23 illustrates a front isometric view of the rectangular shaped interbody spacer device of FIG. 22.

FIG. 24 illustrates a top view of the rectangular shaped interbody spacer device of FIG. 22.

FIG. 25 illustrates a side view of the rectangular shaped interbody spacer device of FIG. 22.

FIG. 26 illustrates an end view the rectangular shaped interbody spacer device of FIG. 22.

### DETAILED DESCRIPTION OF THE INVENTION

In the following description, certain specific details are set forth in order to provide a thorough understanding of various embodiments of the invention. However, one skilled in the relevant art will recognize that the invention may be practiced without one or more of these specific details, or with other methods, components, materials, etc. In other instances, well-known structures associated with intervertebral spacer devices and the spine have not been shown or described in detail to avoid unnecessarily obscuring descriptions of the embodiments of the invention.

Unless the context requires otherwise, throughout the specification and claims which follow, the word “comprise” and variations thereof, such as, “comprises” and “comprising” are to be construed in an open, inclusive sense, that is as “including, but not limited to.”

Reference throughout this specification to “one embodiment” or “an embodiment” means that a particular feature, structure or characteristic described in connection with the embodiment is included in at least one embodiment of the present invention. Thus, the phrases “in one embodiment” or “in an embodiment” in various places throughout this specification do not necessarily all refer to the same embodiment. Furthermore, the particular features, structures, or characteristics may be combined in any suitable manner in one or more embodiments to form additional embodiments.

The headings provided herein are for convenience only and do not interpret the scope or meaning of the embodiments.

According to aspects of this description, an expandable interbody spacer (IBS) device is provided to restore disc height between vertebral bodies without the insertion of height expanding surgical devices. According to one embodiment of the invention, the device is inserted into the disc space in a collapsed or unexpanded position and an expansion member or spreader is engaged to increase the height of the interbody spacer device to an expanded position. Expanding the height of the device by engaging the expansion member will correspondingly expand the height of the disc space to restore the desired interbody spacing between discs.

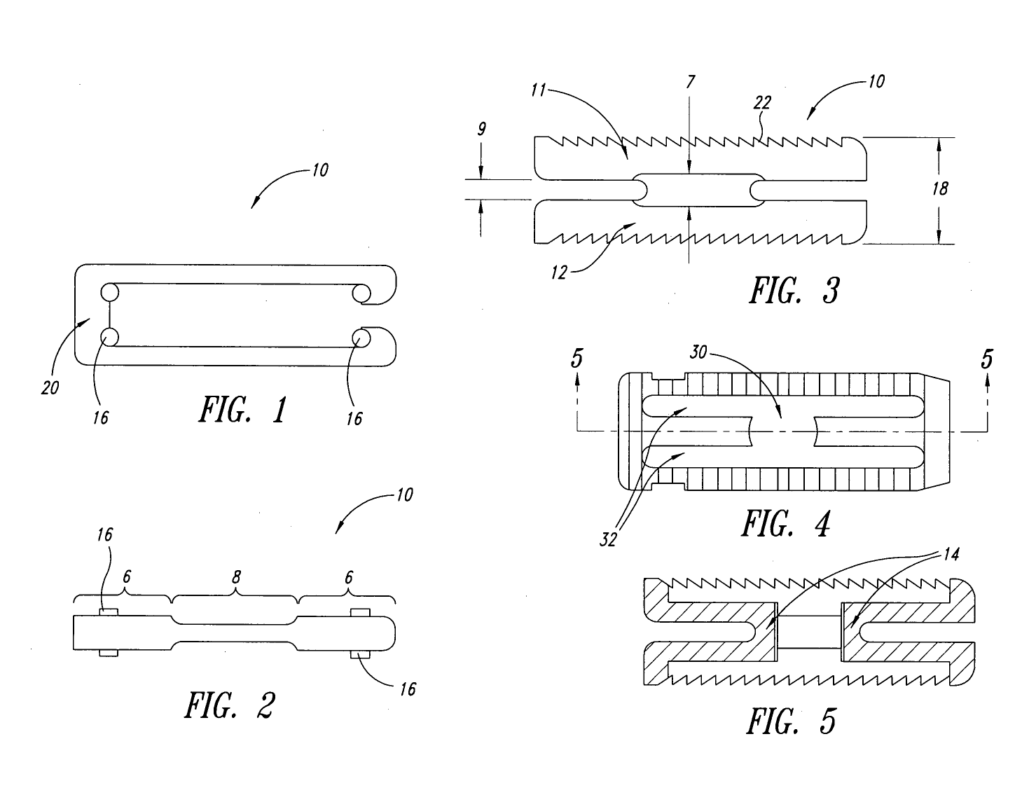

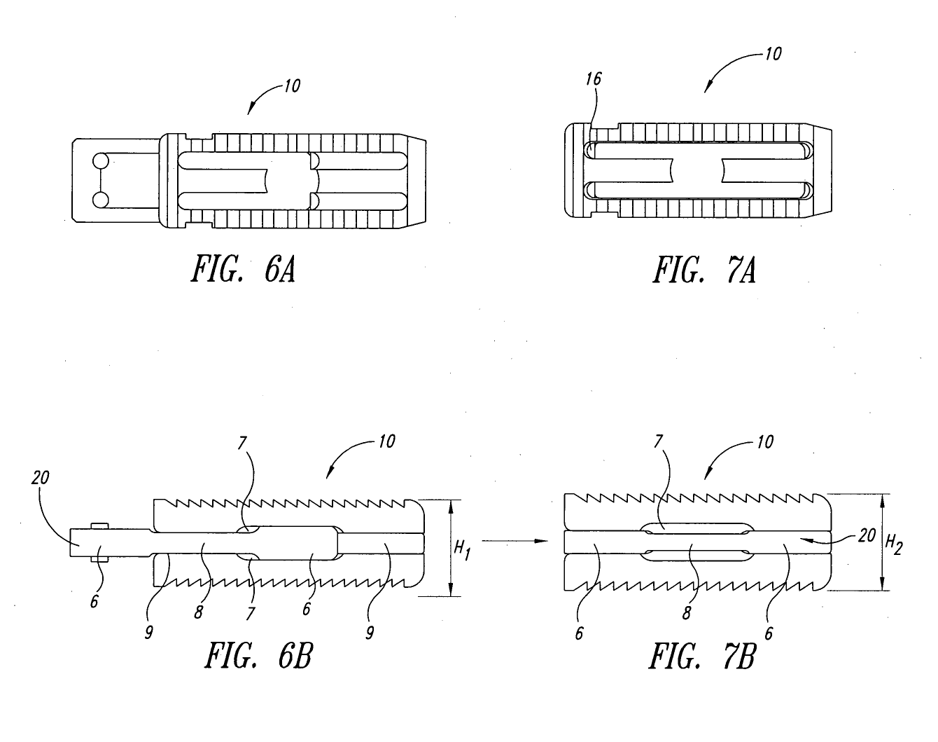

FIGS. 1-5 show an interbody spacer device 10 comprising an expansion member or spreader 20 for positioning between a first planar element or plate 11 and a second planar element or plate 12. The plates 11, 12 are connected by one or more connection members 14 that retain the plates 11, 12 in a position proximate to one another while allowing them to move laterally to expand away from one another.

FIGS. 1 and 2 show one embodiment of a u-shaped expansion member 20. The expansion member 20 includes end sections 6 having a first width and a recessed section 8 provided between the two end sections 6. A longitudinal passageway extending between the two plates 11, 12 has a varying diameter, such that it has a relatively wide central portion 7 and a narrower channel 9 provided on either side of the wide central portion. When the interbody spacer device is in an unexpanded state, a first end section 6 of the spreader 20 is retained in the corresponding wide section 7 of the assembly provided between the first and the second plate 11, 12. The recessed section 8 of the spreader 20 is positioned in the narrower channel 9 formed between the first and the second plate 11, 12. Furthermore, when the expansion member is partially inserted between the plates 11, 12 of the interbody spacer device 10, the interbody spacer remains in an unexpanded configuration. Accordingly, the expansion member 20 may be pre-assembled with the interbody spacer device prior to implantation by sliding the spreader 20 between surface plates 11, 12.

FIGS. 3, 4 and 5 show exemplary views of the plates 11, 12 and the connecting member 14 prior to the insertion of the expansion member 20. According to aspects of this embodiment, the plates 11, 12 further include an outer surface 22 for contacting endplates of the vertebral bodies (not shown). As shown, the outer surface 22 of the plates 11, 12 is a planer, discontinuous surface having a plurality of spaced apart elongated recesses, grooves, or jagged edges to provide a mating surface for retaining the interbody spacer device in position relative to vertebral bodies. Alternatively, the outer surface could be substantially smooth. In accordance with yet another embodiment, alternative fixation mechanisms could be used to retain the interbody spacer device in position relative to the vertebral bodies as is known in the art.

According to further aspects of the invention, the interbody spacer device is machined such that space 30 is left in the center of the interbody spacer device as a grafting port, or to receive BMP and morsalized bone and thus aid in fusion.

FIGS. 6A and 6B show the expansion member 20 and the interbody spacer device 10 assembled in an unengaged or collapsed position. FIGS. 7A and 7B show the interbody spacer device in the engaged or expanded position. More particularly, as the expansion member 20 is moved forward by a user, end section 6 is forced into channel 9. Given that end section 6 has a width greater than a diameter of channel 9, the end section 6 of expansion member 20 forces the plates 11, 12 apart, thereby expanding the interbody spacer device by causing the plates of the interbody spacer device 10 to move apart.

As shown in FIGS. 6B and 7B, the device has a collapsed overall height of H₁ and an overall expanded height of H₂. The increase in height of the device from H₁ to H₂ is due to the insertion of the expansion member to bias the first and second plates apart. In operation, the collapsed device is impacted into the selected disc space and, once in place, the expansion member is engaged to expand the device height. Allowing the device to be implanted in a collapsed form of less height allows easier implantation by the surgeon while minimizing trauma to the disc site.

According to aspects of the invention, the expansion member 20 further includes retaining tabs 16 that engage slots 32 in the interbody spacer device 10 on each side of the connection member 14. The tabs 16 may guide the expansion member 20 into place. Alternatively, the tabs 16 may also serve to lock the expansion member 20 in place when the interbody spacer device 10 is in an expanded position and the expansion member 20 is engaged as shown in FIGS. 7A and 7B.

Transforaminal Lumbar Interbody Fusion (TLIF)

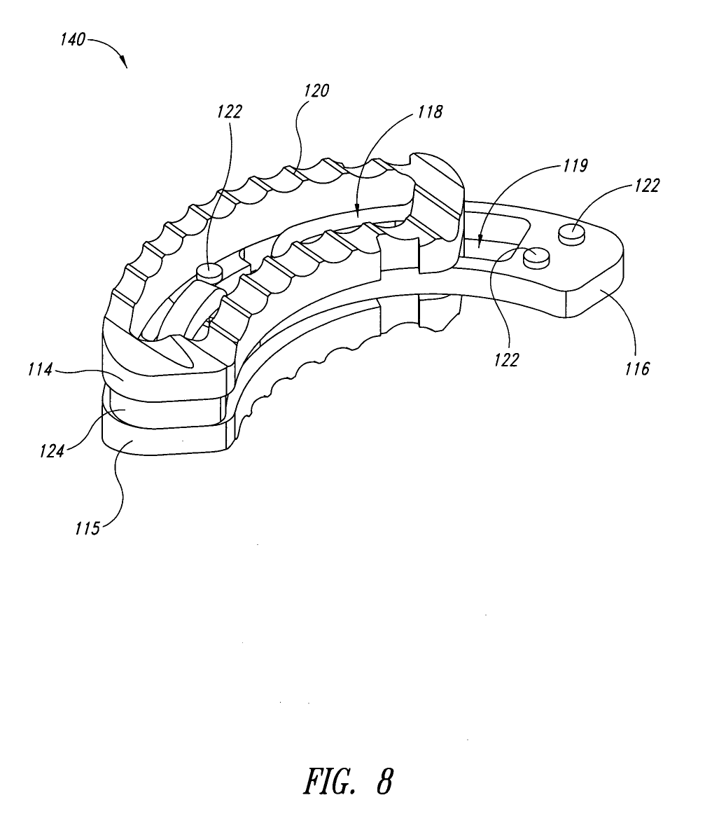

Referring now to FIGS. 8-10, an exemplary intervertebral spacer device 140 is shown. The interbody spacer device 140 replaces a diseased or damaged spinal disc, and more particularly is used in a transforaminal lumbar interbody fusion (TLIF). A TLIF is a posterior and lateral approach to the disc space. Typically the facet joint is removed and access is gained to the disc space via the nerve foramen. While more technically demanding of the surgeon, this approach eliminates the need for manipulation of neural elements, thus reducing the risk of post-operative neural deficit. Furthermore, much of the soft tissue is left intact, placing this technique in the category of less invasive.

Usually, according to this surgical approach, a single implant is placed and is surrounded by bone grafting material (e.g., autograft or BMP). A TLIF implant does not need to be hollow as ample space would be available between the endplates of the vertebral bodies for a fusion mass.

According to known surgical protocol for a TLIF procedure, the implant is placed in the anterior aspect of the disc space, thus providing space for a substantial fusion mass and the creation of normal sagittal alignment (i.e., lordosis). According to one embodiment, a TLIF implant may be cashew or banana shaped, having a tapered leading edge to facilitate its insertion into the disc space. Surface texture (grooves, dimples, surface roughness, spikes and the like) would be oriented to prevent implant migration through the nerve foramen; migration of the implant anteriorly or posteriorly would be prevented by the presence of the surrounding ligaments. In operation, the primary goals of implanting a TLIF interbody spacer device are to immobilize the affected vertebrae, restore the spinal disc space, prove sagittal alignment, and to provide an environment for bony fusion between vertebral bodies.

An oblique surgical approach is~similar to a TLIF surgical approach except for the final placement of the implant; namely, an oblique surgical approach places the implant in the central aspect of the disc space. Graft can be placed anterior and posterior to the implant. An oblique implant may alternatively have a rectangular footprint. Because the implant would lie at an oblique angle across the disc space, in order to restore lordosis, the implant may be positioned such that a tallest edge is at the most anterior corner of the implant and a shortest edge is at the most posterior corner of the implant.

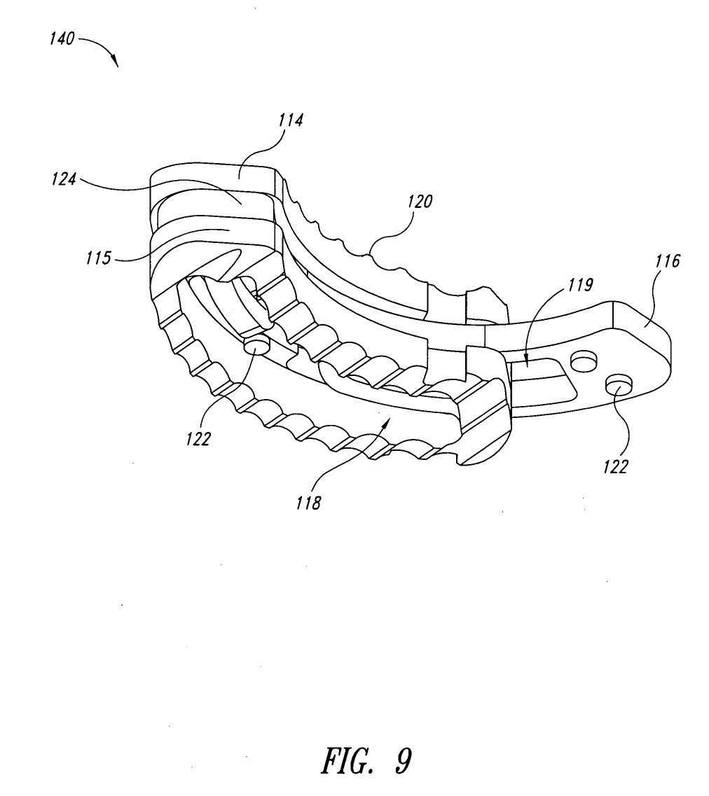

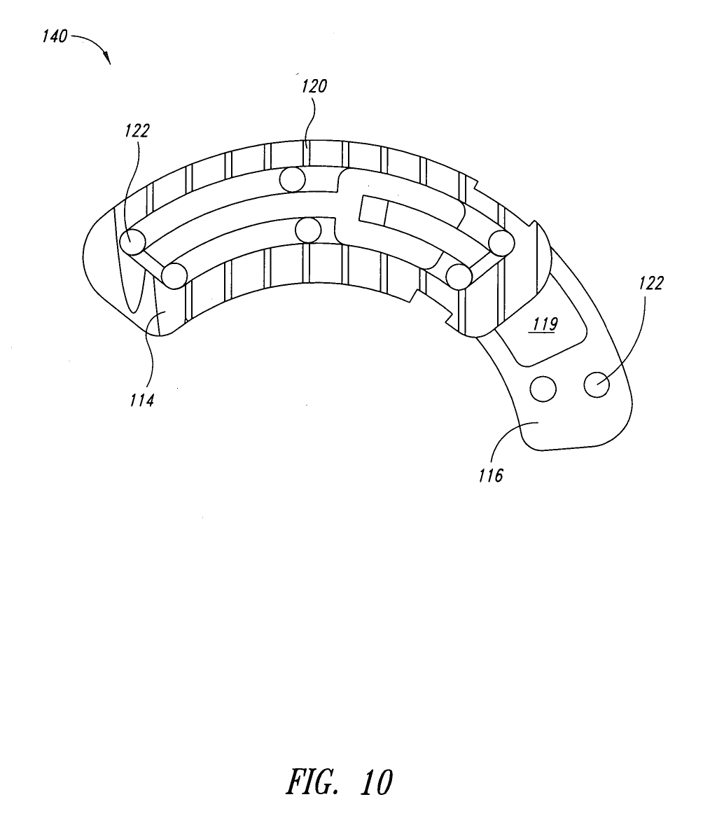

FIGS. 8-10 show exemplary cashew or banana shaped implants, for example, for use with a TLIF approach. More specifically, FIG. 8 shows a top front isometric view of a cashew shaped interbody spacer device for use in a TLIF procedure. FIG. 9 illustrates a bottom isometric view of the cashew shaped interbody spacer device of FIG. 8. FIG. 10 illustrates a top view of the cashew shaped interbody spacer device of FIG. 8.

The cashew shaped interbody spacer device 140 includes a first surface plate 114 and a second surface plate 115 retained in a proximate position by a connection member 124. Alternatively, the first surface place 114 and the second surface plate 115 may be slideably connected to an expansion member 116. The expansion member 116 is sandwiched between the plates 114, 115 and is moveable therebetween. The expansion member 116 moves between a first unexpanded position and a second expanded position causing the interbody spacer device 140 to move between a collapsed position of less overall height and an expanded position of greater overall height. The interbody spacer device 140 of FIGS. 8-10 is shown in an interbody spacer device in an unexpanded position, for example, as the device would be configured prior to implantation.

According to aspects of the embodiment, the expansion member 116 includes tabs 122 for retaining the expansion member in a locked relationship with the plates 114, 115 when the expansion member 116 is engaged such that the interbody spacer device 140 is in an expanded position. According to aspects of the embodiment, the tabs 122 may be fixed protrusions or may be retractable dimples. As shown in the illustrated embodiment, the tabs 122 may be retained in an aperture 118 in the plates. Alternatively, the plates may contain grooves or other alignment guides to align and/or retain the tabs. According to yet another embodiment, the plates 114, 115 may contain the tabs for retaining the expansion member. In accordance with further embodiments, the expansion member 116 may be secured in a locked position relative to the plates by a latch, pin, catch, or other retaining mechanism as is known in the arts.

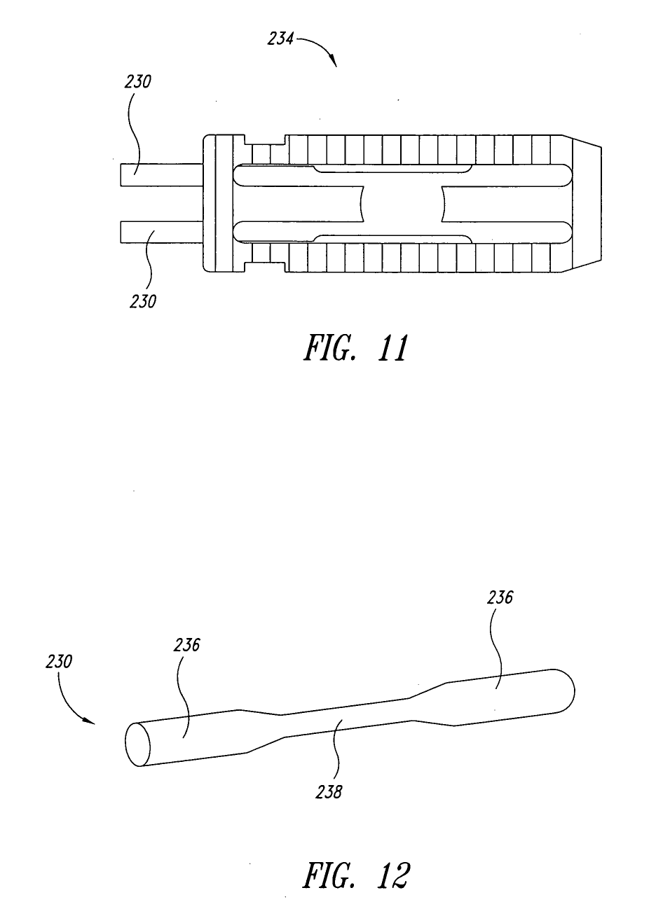

FIG. 11 shows an intervertebral spacer device in a collapsed state prior to extending the expansion member. FIG. 12 shows an expansion member configured as a dowel for use in the interbody spacer device of FIG. 11. As shown in FIG. 11, two dowels or pins 230 are contained in the interbody spacer device 234 to provide a means for spreading the plates and extending the interbody spacer device. As shown in FIG. 12, the dowels 230 include thickened ends 236 and a thinner, or recessed center portion 238. When the dowel is placed in an unengaged or unexpanded-position, a first thickened end 236 resides in a recess in the interbody spacer device to allow the interbody spacer device 234 to maintain a collapsed state.

FIG. 13 shows an alternative embodiment of an interbody spacer device having a lordodic angle L in accordance with principles of the present invention. As shown in FIG. 13, the interbody spacer device has a taller first edge, as compared to a second edge. For one embodiment, the anterior edge is taller than the posterior edge. Thus, the planar faces of the interbody spacer device plates are diverging to aid in restoring lordosis. Alternatively, lordosis can be attained via a tapered expansion member or clip and a constant plate thickness, or a combination of a tapered expansion member and one or both of a tapered plate.

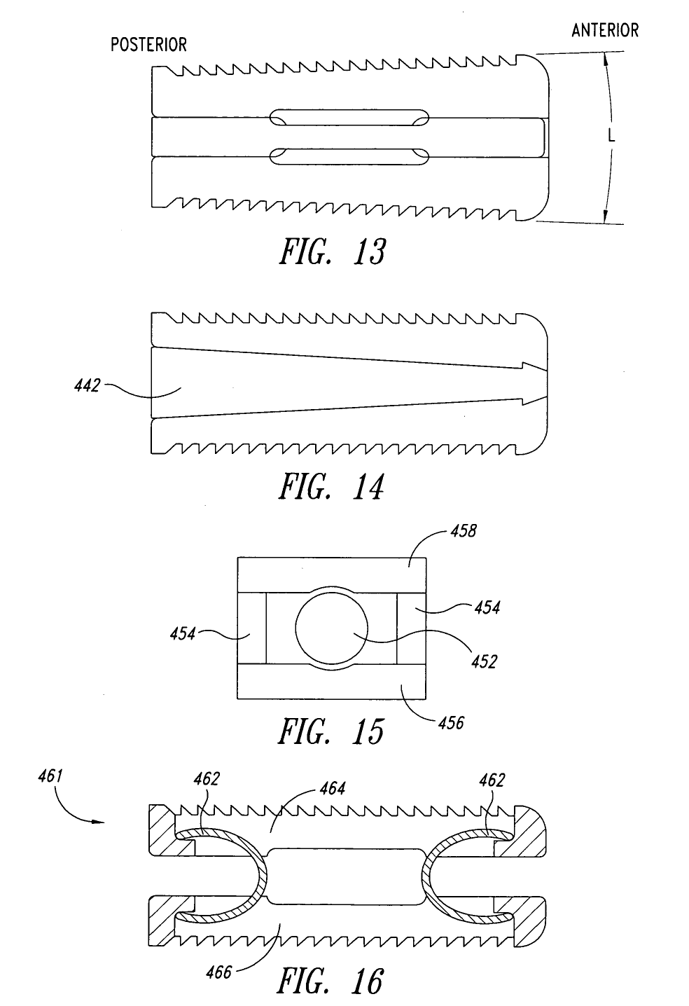

For example, FIG. 14 shows an alternative embodiment of an interbody spacer device having wedge-shaped expansion member 442. According to further aspects, the expansion member may be tapered, such as a shim or any angled spreading means for creating a taller anterior edge as compared to the posterior edge when the expansion member is engaged and the interbody spacer is in an expanded position.

FIG. 15 shows an alternative embodiment of an interbody spacer device having a expansion member 452 aligned in the center of the interbody spacer device and connection elements 454 aligned along outer edges of the interbody spacer device to couple a first plate 456 to a second plate 458.

FIG. 16 shows an alternative embodiment of an interbody spacer device having separate bias elements 462 as the connection elements. According to aspects of this embodiment, a first plate 464 and a second plate 466 are flexibly retained in a position proximate to one another, for example, in a substantially parallel position relative to each other, by bias elements 462. The bias elements 462 may be a spring, c-shaped clamp, clamp, coil, clip or other connection element for retaining the first 464 and second 466 plate of the interbody spacer device 461 in a relative position while allowing the plates to move away from each other when a expansion member is inserted between the plates of the interbody spacer device as described further above.

Anterior Lumbar Interbody Fusion

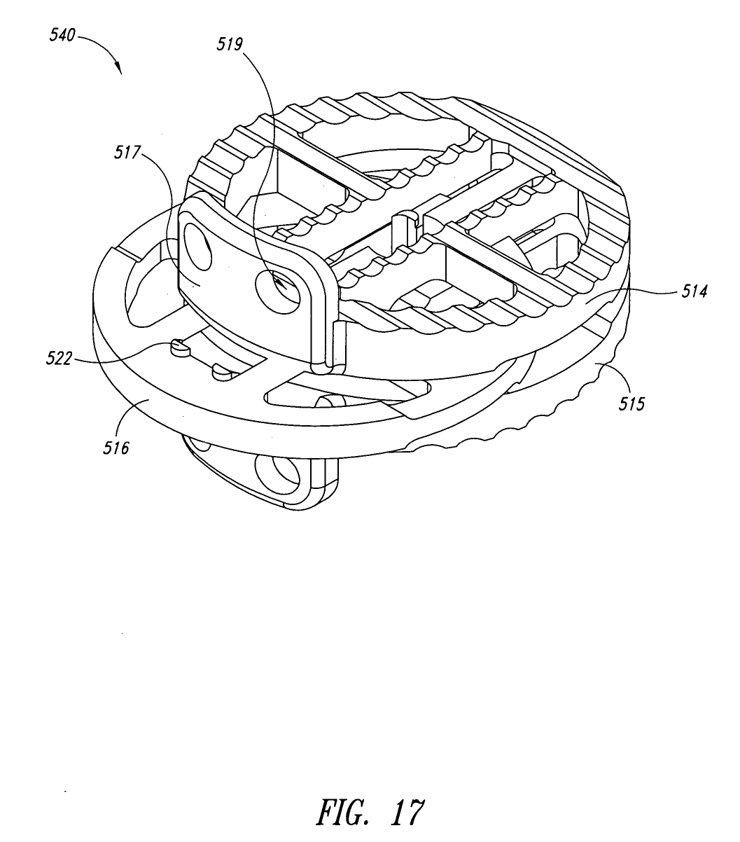

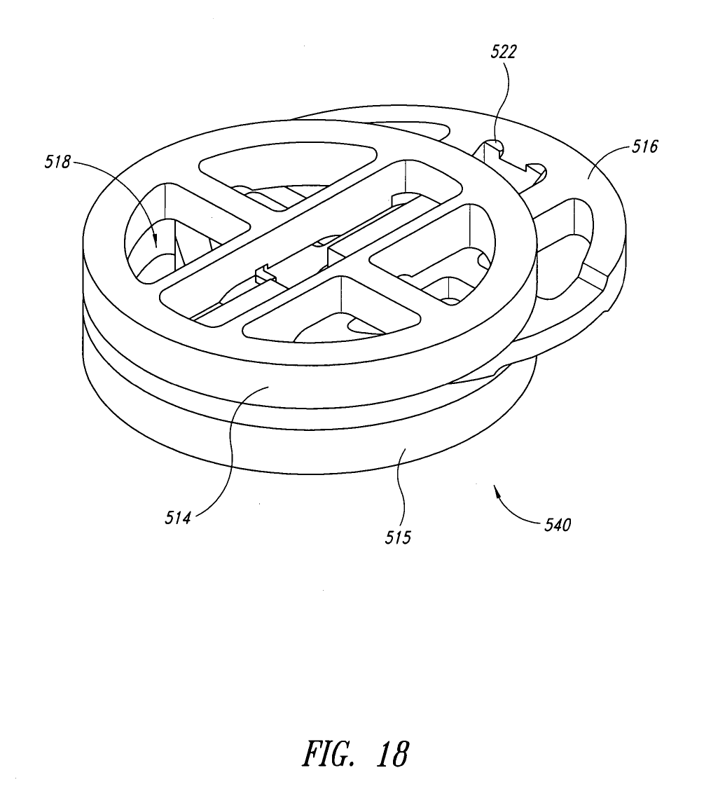

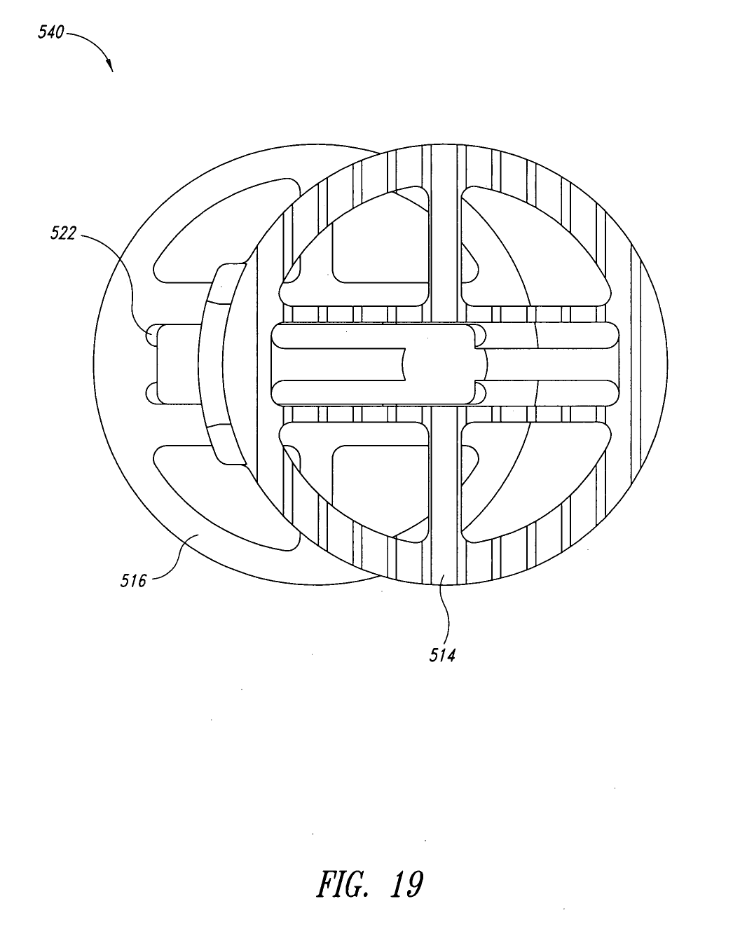

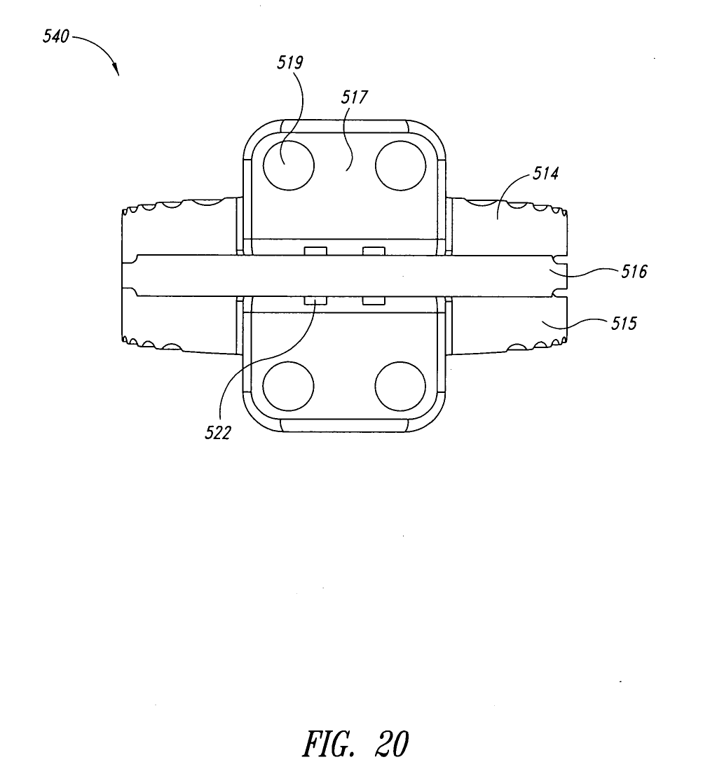

Referring now to FIGS. 17-21, an exemplary interbody spacer device 540 is shown. The interbody spacer device 540 replaces a diseased or damaged spinal disc, and more particularly, is used in an anterior or cervical lumbar interbody fusion. Anterior Lumbar Interbody Fusion (ALIF) is an anterior approach to the disc space. A second, general surgeon is often employed to gain access through the abdominal cavity to the anterior aspect of the spine. The anterior vessels are mobilized and the anterior longitudinal ligament is excised. Access to the posterior neural elements is not attained.

An ALIF is more risky in aged patients or those with sclerotic blood vessels. The cost/need for a second surgeon can be a hindrance. Still, in cases of extremely collapsed disc spaces with little neural stenosis, the approach is ideal.

A large, single implant may typically be used for an anterior approach. The implant is usually hollow and is the size and shape of the adjacent vertebral bodies. With respect to anterior and cervical lumbar interbody fusion, the implant or interbody spacer device differs in regard to the diameter of the interbody spacer device used. The implant is typically packed with and surrounded by bone grafting material, for example, autograft or BMP.

More-specifically, FIG. 17 shows an annular shaped interbody spacer device for use, for example, in an ALIF or cervical procedure. According to alternative embodiments of the invention, the interbody spacer device may be circular, oblong or disc shaped. The interbody spacer device 540 includes a first surface plate 514 and a second surface plate 515 coupled together by a connection member 524. Alternatively, the first surface plate 514 and the second surface plate-515 are coupled directly to the expansion member 516. An expansion member 516 is sandwiched between the plates 514, 515 and is moveable therebetween. The expansion member moves the interbody spacer device from a first unexpanded position to a second expanded position. The anterior or cervical interbody spacer device of FIGS. 17-21 is shown in an unexpanded position such as prior to implantation in the interbody spacer device.

As shown in FIGS. 17, 19, 20 and 21, a tab 517 on a superior edge of the interbody spacer device includes an aperture 519 which may be used to attach the interbody spacer device to the vertebral bodies with screws, staples, pins or the like. Alternatively, a flange, loop, or other fixation means contained on the interbody spacer device may be used to attach the interbody spacer device to the vertebral bodies. Alternatively, as shown in FIG. 18, the device may be provided without tab 517.

According to aspects of this invention, the expansion member 516 includes tabs 522 for retaining the expansion member in a locked relationship with the plates 514, 515 when the expansion member is engaged to place the interbody spacer device in an expanded position. Alternatively, the plates may contain tabs for retaining the expansion member. In accordance with further embodiments of the present invention, the expansion member could be secured in a locked position relative to the plates by a latch, pin, catch, or other retaining mechanism as is known in the arts.

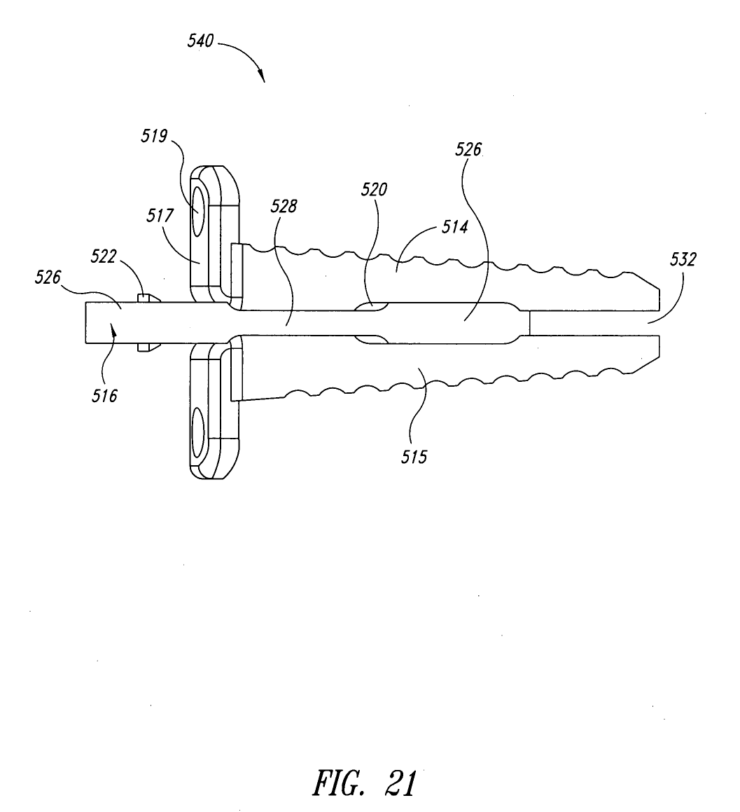

As shown in FIG. 21, the expansion member 516 includes end sections 526 having a first width and a recessed-section 528 provided between the two end sections 526. A longitudinal passageway extending between the two plates 514, 515 has a varying diameter, such that it has a relatively wide central portion 530 and a narrower channel 532 provided on either side of the wide central portion. When the interbody spacer device is in an unexpanded state, a first end section 526 of the expansion element 516 is retained in the corresponding wide section 530 of the assembly provided between the first and the second plate 514, 515. The recessed section 528 of the expansion element 516 is positioned in the narrower channel 532 formed between the first and the second plate 514, 515. Furthermore, when the expansion member is partially inserted between the plates of the interbody spacer device, the interbody spacer remains in an unexpanded configuration. Moving the expansion member to a position between the plates of the interbody spacer causes the wider end sections 528 of the expansion member 516 to push the plates 514, 515 apart, thus expanding the interbody device.

As further shown in FIG. 21, the plates 514, 515 are tapered to create lordosis. Alternatively, lordosis can be attained via a tapered expansion member 516 or clip and a constant thickness plate, or a combination of a tapered expansion member and one or both of a tapered plate as described further herein. For example, the expansion member or clip may be tapered, such as a wedge shape or other angled spreading means for creating a taller anterior edge as compared to the posterior edge when the expansion member is in the engaged or expanded position, and the thickness of the plates can remain constant.

Lateral and Posterior Lumbar Interbody Fusion Device

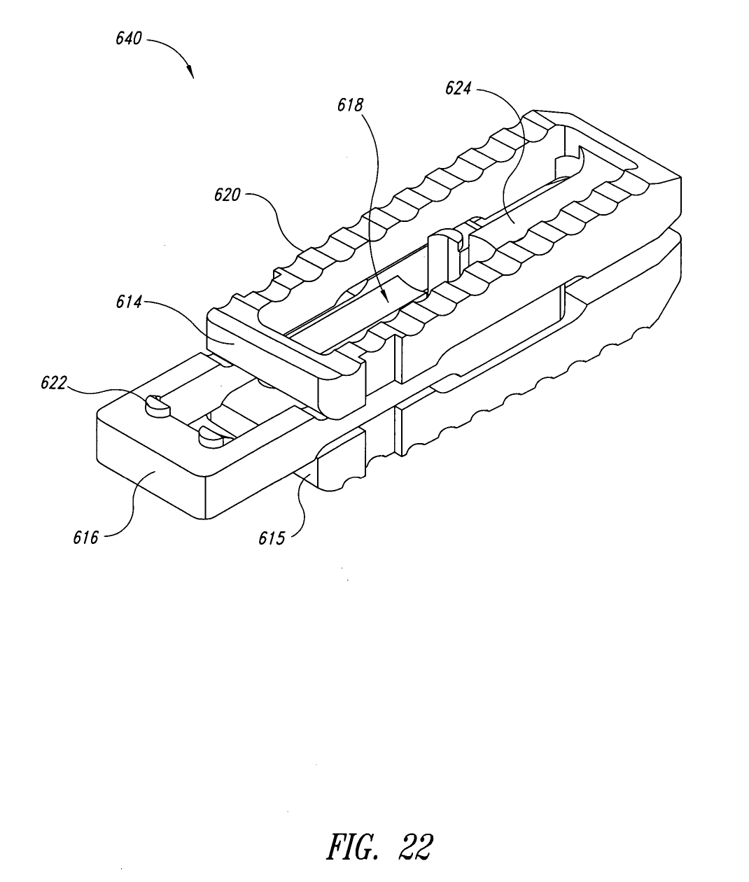

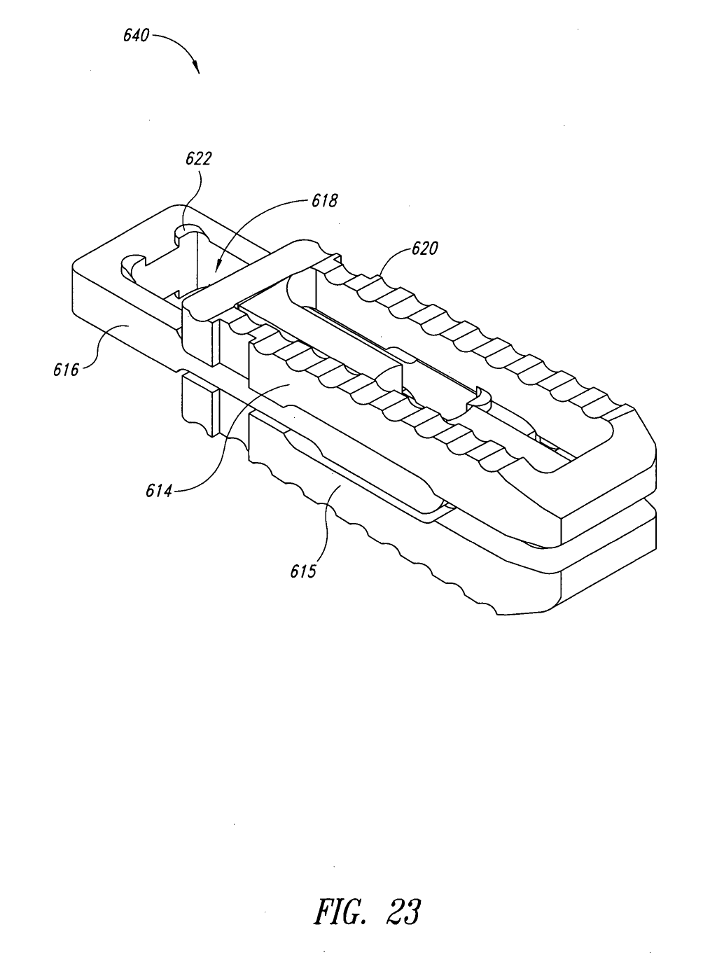

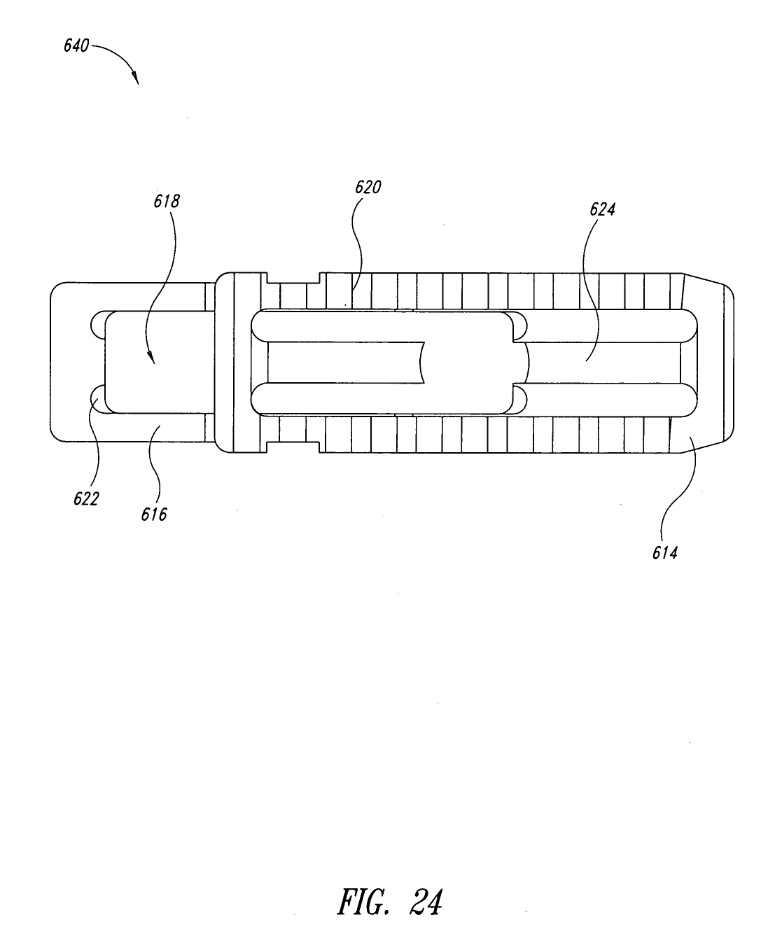

Referring now to FIGS. 22-26, an exemplary interbody spacer device 640 is shown. The interbody spacer device 640 replaces a diseased or damaged spinal disc, and more particularly, is used in a posterior or lateral lumbar interbody fusion. A posterior lumbar interbody fusion (PLIF) is a posterior and midline approach to the disc space. Typically portions of the lamina are removed. The ligamentum flavum and posterior longitudinal-ligament are excised. The spinal cord/deural sac is mobilized to provide access to the disc space.

While it is more commonly practiced and is less technically demanding, a PLIF approach poses greater risk to the patient than does, for example, a TLIF technique; manipulating neural elements creates the potential for damage to them. Traditionally, two implants are placed, one to each side of the midline. For thread-into-place implants, the shape is usually cylindrical. For impact-into-place implants, the shape is usually rectangular. Rectangular implants decrease the distance that the deura is moved by having a height to width ratio greater than 1 and therefore are preferable.

A PLIF implant is often hollow to allow additional space for bone grafting material. The use of two implants decreases the amount of disc space left for placement of bone grafting material, thus the hollow implant cavity provides additional space for bone grafting. Implants typically have an anterior to posterior taper to provide for proper sagittal alignment of the spine. The superior and inferior surfaces may be convex to increase the intimacy of the implant mate with the endplates of the vertebrae. Surface texture is typically configured to prevent posterior implant migration.

A lateral approach to interbody fusion is similar to a PLIF, except the approach is orthogonal to a PLIF approach. Two implants are still used. The implants can be cylindrical thread-into-place implants or rectangular impacted implants. As two implants are most commonly placed, little space is left for grafting, which requires that the implants be hollow for graft placement. To restore lordosis the implants would typically taper from the anterior side to the posterior side.

More specifically, FIGS. 22-26 show a rectangular shaped interbody spacer device for use in a lateral, oblique or PLIF procedure. According to alternative embodiments of the invention, the interbody spacer device may be square or polygonal shaped.

The interbody spacer device 640 includes a first surface plate 614 and a second surface plate 615 coupled together by a connection member 624. Alternatively, the first surface plate 614 and the second surface plate 615 may be slideably connected directly to an expansion member 616. According to yet another alternative embodiment described herein, the expansion member 616 may be a bias element such as a clip, spring or clamp. As shown in FIG. 22, an expansion member 616 is positioned between the plates 614, 615 and is moveable therebetween.

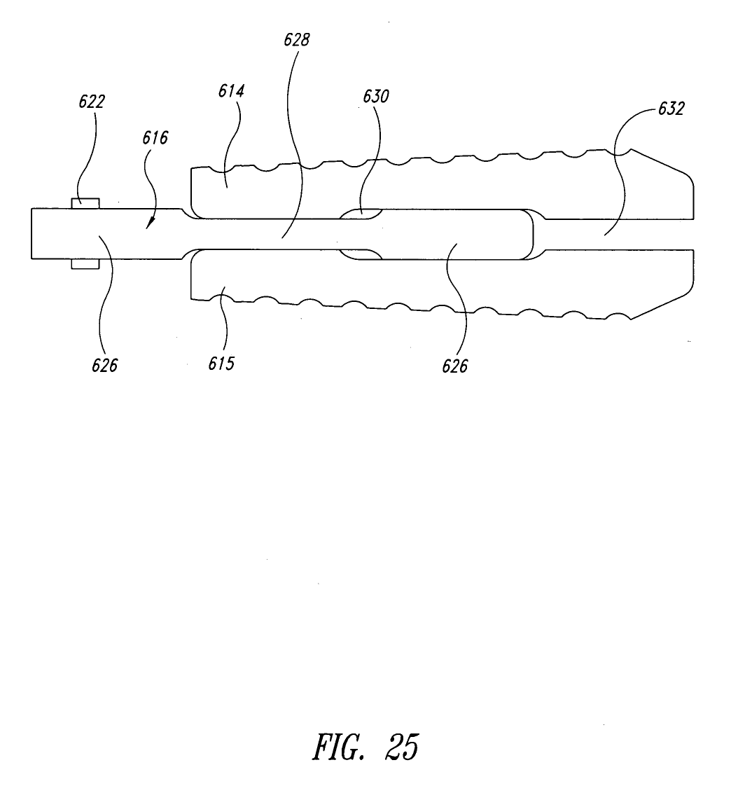

As shown in FIG. 25, the expansion member 616 includes end sections 626 having a first width and a recessed section 628 provided between the two end sections 626. A longitudinal passageway extending between the two plates 614, 615 has a varying diameter, such that it has a relatively wide central portion 630 and a narrower channel 632 provided on either side of the wide central portion. When the interbody spacer device is in an unexpanded state, a first end section 626 of the expansion element 616 is retained in the corresponding wide section 630 of the assembly provided between the first and the second plate 614, 615. The recessed section 628 of the expansion element 616 is positioned in the narrower channel 632 formed between the first and the second plate 614, 615. When the expansion member is partially inserted between the plates of the interbody spacer device, the interbody spacer remains in an unexpanded configuration. Moving the expansion member to a fully engaged position between the plates of the interbody spacer causes the wider end sections 628 of the expansion member 616 to push the plates 614, 615 apart, thus expanding the interbody device. Therefore, the expansion member moves between a first unexpanded position to a second expanded position. The anterior or cervical interbody spacer device of FIGS. 22-26 is shown in an unexpanded position such as prior to implantation in the interbody spacer device.

According to aspects of this invention, the expansion member 616 includes tabs 622 for guiding the expansion member between the plates and/or for retaining the expansion member in a locked relationship with the plates 614, 615 when the expansion member is fully inserted between the plates. Alternatively, the plates may contain tabs for retaining the expansion member. In accordance with further embodiments of the present invention, the expansion member could be secured in a locked position relative to the plates by a latch, pin, catch, or other retaining mechanism as is known in the arts.

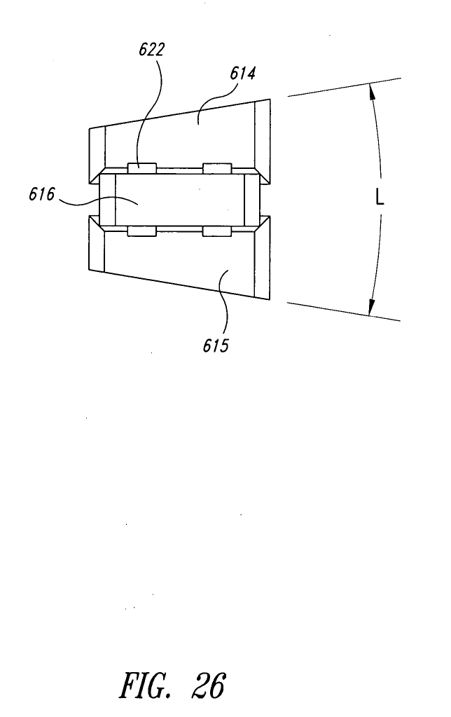

As shown in FIG. 26, the plates 614, 615 are tapered to create lordosis. Alternatively, lordosis can be attained via a tapered expansion member or clip and a constant: interbody spacer device, or a combination of a tapered expansion member and a tapered interbody spacer device as described further herein.

According to aspects of the invention, the interbody spacer devices provided in accordance with the present invention may be made of a variety of materials, including but not limited to: stainless steel, carbon fiber materials, various plastics, titanium, ceramic, PEEK, or bio-absorbable materials. The material may be non-porous, inert and biologically compatible. The material may further be of such character as to form a rigid, non-resilient load-bearing material, one that is preferably incapable of elastic deformation.

The components of the interbody spacer device, such as the plates and the expansion member described herein, can be machined and/or molded to provide the features disclosed. The components of the interbody spacer device may be of the same material, or different materials.

As discussed herein, and in accordance with alternative embodiments of the invention, the configuration of the interbody spacer device may have parallel faces, but could also be produced with angled faces in a variety of orientations to restore lordosis with different orientations of the device within the disc space. In accordance with one embodiment of the invention, the interbody spacer device could also be configured such that engaging the device expands only one end to reproduce a lordodic angle. In accordance with an alternative embodiment of the invention, the interbody spacer device has a convex anterior sidewall and a concave posterior sidewall, thus allowing a concave to convex contour with respect to a plane across the spacer device. The interbody spacer device according to one aspect is cashew shaped, to accommodate a transforaminal lumbar interbody fusion surgical approach. According to alternative embodiments of the invention, the interbody spacer may be square, polygonal or rectangular shaped.

Several advantages are evident with respect to the interbody spacer device disclosed herein. By allowing the interbody spacer-device to be inserted in a collapsed or unexpanded state, the surgeon is able to place the spacer device without over retracting the wound site. Once in place, the spacer device can be engaged, causing the interbody spacer to attain an expanded position to allow full restoration of the spinal disc space with minimal impact to the vertebral bodies.

The above description of illustrated embodiments, including what is described in the Abstract, is not intended to be exhaustive or to limit the invention to the precise forms disclosed. Although specific embodiments of and examples are described herein for illustrative purposes, various equivalent modifications can be made without departing from the spirit and scope of the invention, as will be recognized by those skilled in the relevant art. The teachings provided herein of the invention can be applied to intervertebral spacer devices, not necessarily the exemplary cashew shaped transforaminal spacer devices generally described above.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet in their entirety. Aspects of the invention can be modified, if necessary, to employ systems, materials and concepts of the various patents, applications and publications to provide yet further embodiments of the invention.

These and other changes can be made to the invention in light of the above-detailed description. In general, in the following claims, the terms used should not be construed to limit the invention to the specific embodiments disclosed in the specification and the claims, but should be construed to include all intervertebral spacer devices that operated in accordance with the claims. Accordingly, the invention is not limited by the disclosure, but instead its scope is to be determined entirely by the following claims.

1. A spinal interbody spacer device comprising:
an implant body having a first plate coupled to and spaced apart from a second plate; and
an expansion member coupled between the plates and moveable from a first position to a second position, the expansion member exerting a force on the first and second plates when in the second position to increase a height of the interbody spacer device.

2. The spinal interbody spacer device of claim 1 wherein the expansion member is a wedge.

3. The spinal interbody spacer device of claim 1 wherein the expansion member is a pin.

4. The spinal interbody spacer device of claim 1 wherein the connection member is a c-shaped clamp.

5. The spinal interbody spacer device of claim 1 wherein the first plate, second plate and the expansion member are all made from titanium.

6. The spinal interbody spacer device of claim 1, further including grooves on the outer surface of the plates, wherein the grooves are configured to mate with endplates of vertebral bodies.

7. The spinal interbody spacer device of claim 1 wherein the expansion member is slideably moveable.

8. The spinal interbody spacer device of claim 1 wherein an anterior side of the plates is taller than a posterior side of the plates, thus providing a lordodic angle.

9. The expandable intervertebral spacer device of claim 1 wherein an anterior side of the spreader is taller than a posterior side of the spreader.

10. A spinal intervertebral spacer device, comprising:
a first planar element;
a second planar element spaced apart from the first planar element;
a connection element coupling the first planar element to the second planar element, wherein the connection element retains the first planar element spaced apart from the second planar element; and
a spacer device positioned between the first and second planar elements, wherein the spacer device is moveable between the planar elements.

11. The expandable intervertebral spacer device of claim 10 further comprising:
a tab contained on an outer surface of the spacer device; and
a reciprocal receiving groove provided opposite the tab on an adjacent surface of the first planar element, wherein the tab engages the receiving groove and retains the spacer device in a selected position.

12. The expandable intervertebral spacer device of claim 10 wherein the spacer device is a dowel.

13. The expandable intervertebral spacer device of claim 10 wherein the spacer device is a unshaped clip.

14. The expandable intervertebral spacer device of claim 10 wherein the spacer device is laterally engaged to move the first surface element and the second surface element apart from each other.

15. The expandable intervertebral spacer device of claim 10 wherein the first and second surface element, the connection element and the spacer device are all made from a biologically compatible, inert material.

16. The expandable intervertebral spacer device of claim 10 wherein an anterior edge of the spacer device is taller than a posterior edge of the spacer device.

17. The expandable intervertebral spacer device of claim 10 wherein an anterior edge of the spacer is taller than a posterior edge of the spreader.

18. A spinal intervertebral spacer device, comprising:
a first disc shaped element;
a second disc shaped element proximate to the first disc shaped element, the first and the second disc shaped element having a spacing therebetween; and
a spacer device juxtaposed between the first and second element, wherein the spacer device is moveable between the elements, the spacer device having a first side and a second side, the first side and the second side having a connection mechanism affixed to the first element and to the second element, wherein the connection mechanism slideably retains the first element and the second element on the spacer device, wherein the first element, the second element and the spacer device combine to provide intervertebral support when implanted in a spine.

19. A method of implanting a spinal interbody spacer device, comprising:
impacting an expandable interbody spacer device into a lumbar region of the spine; and
engaging an integral spreader to expand the implant, wherein engaging the spreader includes moving the spreader between a first and a second element of the implant causing the first and the second element to move apart from one another.

20. The method of claim 19 further comprising:
packing at least one of an aperture of the expandable interbody spacer device with BMP.

21. The method of claim 19 wherein the spreader is laterally engaged.
